(19) United States
(12) Patent Application Publication
(10) Pub. No.: US 2022/0257801 A1
(43) Pub. Date: Aug. 18, 2022

(54) PARTICULATE STRUCTURES MADE FROM GOLD NANOPARTICLES, METHODS FOR PREPARING SAME AND USES THEREOF FOR TREATING SOLID TUMOURS

(71) Applicant: UNIVERSITE DE FRANCHE COMTE, BESANCON (FR)

(72) Inventors: Gautier LAURENT, POUILLEY-LES-VIGNES (FR); Arnaud BEDUNEAU, Valdahon (FR); Stéphane ROUX, CHENEYCEY BUILLON (FR)

(73) Assignee: UNIVERSITE DE FRANCHE COMTE, BESANCON (FR)

(21) Appl. No.: 17/625,245

(22) PCT Filed: Jul. 23, 2020
(86) PCT No.: PCT/FR2020/051352

§ 371 Date: Jan. 6, 2022

(30) Foreign Application Priority Data
Jul. 23, 2019 (FR) 19 08368

(51) Int. Cl. A61K 49/04; A61K 49/18

(52) U.S. Cl. CPC A61K 49/0428; B82Y 15/00; A61K 49/1881

(57) ABSTRACT

A particulate structure that includes a/ a biodegradable polymer particle, b/ gold nanoparticles covered on their surface with macrocyclic chelators complexing at least one ion of interest and/or a radionuclide for medical imaging, c/ a polycation having a positive charge over a pH range from 5 to 11, the gold nanoparticles b/ being encapsulated in the polymer particle a/ and/or adsorbed at the surface of the polymer particle a/. Also, a method for preparing the particulate structures. Further, the use of the particulate structures for radiotherapy or chemotherapy in the context of cancer treatment.

### TECHNICAL FIELD

The present invention relates to the field of chemistry and formulation applied to health. It relates in particular to new particulate structures that comprise multifunctional gold nanoparticles, and uses thereof for radiotherapy, imaging and chemotherapy in the context of cancer treatment.

The invention also relates to a method for preparing these new particulate structures, which consists in particular of encapsulating the multifunctional gold nanoparticles in biodegradable polymer particles.

### PRIOR ART

The use of gold nanoparticles represents a promising strategy in cancer diagnosis and treatment (1), (2).

The reduced size of the nanoparticles allows exploration of living organisms down to the cellular level. These nanoparticles are large enough for them not to cross the biological barriers of healthy tissues and small enough to cross the porous epithelium of the blood vessels of solid tumors.
Gold nanoparticles are also attractive owing to their intrinsic properties. In fact, the element gold is a noble metal par excellence, which is very insensitive to external chemical aggressive conditions, and furthermore has suitable biocompatibility for medical applications. Gold nanoparticles possess optical properties that can be modulated depending on the size, shape and the dielectric environment. This property is much used in the context of photothermal therapy and imaging (3). Moreover, owing to its high atomic number, gold is characterized by very high density and effective cross section of absorption of X and γ photons. This property, regardless of the size, endows gold nanoparticles with contrast agent behavior for X-ray tomodensitometry and a radiosensitizing effect that is exploitable for radiotherapy (4), (5). Finally, the two main methods of synthesis described by Brust and Frens are relatively easy to implement. The first consists of reduction of a gold salt with a strong reducing agent in the presence of thiolated ligands while Frens' method leads to the formation of nanoparticles stabilized with citrate ions using the reducing agent sodium citrate on the gold salt (6), (7). Functionalization of these gold nanoparticles, which may be carried out during or after synthesis, makes it possible to enrich the range of properties. With a suitable choice of the constituents used for the synthesis of multifunctional gold nanoparticles, it is then possible, despite the reduced size, to integrate therapeutic activity and imaging functions within one and the same object.

The thesis of G. Laurent (8) showed that multifunctional gold nanoparticles, namely gold nanoparticles covered on their surface with macrocyclic chelating agents capable of complexing elements of interest for medical imaging (Gd³⁺ for MRI, ¹¹¹In³⁺ for SPET and ⁶⁴Cu²⁺ for PET), have considerable potential for image-guided radiotherapy.

These gold nanoparticles, thus functionalized, in fact have potential as a multimodal contrast agent (MRI, nuclear imaging) and as a radiosensitizing agent. Once injected intravenously, these nanoparticles have displayed a significant therapeutic effect following their activation with X-rays. Moreover, the biodistribution of these nanoparticles could be monitored by MRI, SPECT and X-ray tomodensitometry. Multifunctional gold nanoparticles, owing to their optical and radiosensitizing properties, therefore represent an extremely interesting approach for tumor diagnosis and treatment.
However, despite these promising results, the plasma half-life of these multifunctional gold nanoparticles is still very short, thus hampering their accumulation in tumoral zones (level of accumulation about 2%). The excessively rapid elimination of the nanoparticles (renal clearance) can be explained by their small size (hydrodynamic diameter of about 6 to 7 nm), but this is crucial to being able to be eliminated by the renal route.

It was thus proposed to increase the hydrodynamic diameter of the gold nanoparticles in order to limit the problem of the excessively quick renal clearance. However, such an approach greatly reduces the radiosensitizing properties of the nanoparticles, as well as their elimination from the body by the renal route (9), (10).

The present inventors then came up with the idea of encapsulating multifunctional gold nanoparticles in larger biodegradable polymer particles, which would circulate in the blood for a longer time and would therefore have more opportunity to accumulate in the tumor, while maintaining renal elimination of the nano-objects.

Approaches for encapsulating gold nanoparticles in biodegradable polymer particles have already been described in the literature. Thus, we may mention encapsulation with a simple oil-in-water emulsion or with a water-in-oil-in-water double emulsion described in Wang Y et al. (11) or else synthesis of gold nanoparticles in situ, namely directly inside the polymer particles, described in Luque-Michel et al. (12).

However, these methods have the drawback of a low encapsulation yield and/or they lead to particles that are too large, namely of the order of a micrometer, and also have the drawback of a lack of uniformity of size (polydisperse particles). Moreover, the gold particles generally encapsulated are “bare” (i.e. not functionalized), which leads to a lack of colloidal stability in the physiological environment and a problem of elimination during degradation of the polymer particle, in vivo.

### SUMMARY

One of the aims of the present invention is to develop new particulate structures that comprise multifunctional gold nanoparticles, and that have a long enough plasma half-life (namely from 15 minutes to 120 minutes) to improve their accumulation at the level of the tumoral zone and better exploit the radiosensitizing potential of the multifunctional gold nanoparticles.

Another aim of the invention is to develop new particulate structures that are biodegradable transporters, which have a plasma half-life (circulation time in the blood) that is long enough for fully exploiting the promising potential of multifunctional gold nanoparticles for image-guided radiotherapy.
Another aim of the invention is to develop new particulate structures which, while having a plasma half-life that is long enough for improving their tumoral accumulation, then degrade rapidly in the blood and are eliminated by the renal route.

Another aim of the present invention is to develop an original method for preparing these new particulate structures, said method allowing efficient encapsulation of multifunctional gold nanoparticles in biodegradable polymer particles, i.e. with an encapsulation yield greater than 90%, close to 100%, or even equal to 100%.

Another aim of the invention is to develop a method for preparing particulate structures of the order of a nanometer, i.e. which have a diameter from 50 to 200 nm, and that have a narrow size distribution (i.e. that have a low polydispersity index).
Another aim of the invention is to develop a method for preparing particulate structures as defined above, with good reproducibility, both for the loading obtained (encapsulation rate) and for the particle size obtained.

In their research concerning the synthesis of biodegradable polymer particles encapsulating gold nanoparticles, the inventors were interested in particular in methods based on the method of nanoprecipitation by solvent displacement (13).

They thus discovered, surprisingly, that the method for preparing these particulate structures could be greatly improved by using a polycation having a positive charge over a wide range of pH, namely a pH range from 5 to 11.
In fact, the polycation makes it possible to trap the multifunctional gold nanoparticles electrostatically, which facilitates and in particular makes possible their encapsulation in the biodegradable polymer particles.

The present invention relates more particularly to a particulate structure, characterized in that it comprises:

a/ a biodegradable polymer particle,
b/ gold nanoparticles covered on their surface with macrocyclic chelating agents complexing at least one ion of interest and/or a radionuclide for medical imaging,
c/ a polycation having a positive charge over a pH range from 5 to 11,
the gold nanoparticles b/ being encapsulated in the polymer particle a/ and/or adsorbed on the surface of the polymer particle a/.

The term “nanoparticle” denotes an object, of whatever shape, at least one dimension of which is between 1 and 100 nanometers.

The particulate structure of the invention denotes in particular a biodegradable polymer particle a/, inside which gold nanoparticles b/ are encapsulated and/or on the surface of which gold nanoparticles b/ are adsorbed.

Regarding the gold nanoparticles, the possible shapes may be spheres, nanoshells (core-shell), nanorods. However, the spherical shape is an approximation. In fact, gold crystallizes in a face-centered cubic lattice and thus forms a polyhedral object that may be likened to a sphere.

According to the invention, the gold nanoparticles and the biodegradable polymer particles are preferably of spherical shape. Similarly, the particulate structure of the invention is preferably of spherical shape.

The gold nanoparticles of the particulate structures of the invention, covered on their surface with macrocyclic chelating agents complexing at least one ion of interest and/or a radionuclide for medical imaging, may also be referred to by any of the terms “functional” gold nanoparticles (as opposed to “bare” gold nanoparticles), “multifunctional”, “functionalized”, radiosensitizing functionalized gold nanoparticles etc. They may simply be designated hereinafter as gold nanoparticles b/. These gold nanoparticles b/ thus consist of a gold core surrounded or covered with an organic layer consisting of macrocyclic chelating agents complexing ions of interest and/or radionuclides.

The essential role of the organic layer, besides colloidal stability, is to allow complexation of the elements for medical imaging (ion of interest, radionuclide) so as to be able to track the gold nanoparticles b/ by imaging.

The functional gold nanoparticles b/ may also be denoted by the symbol Au@L(M), in which Au represents gold, and L(M) represents the macrocyclic chelating agent (namely L) complexing the ion of interest and/or the radionuclide (namely M).

The macrocyclic chelating agent L may also be denoted by macrocyclic ligand or ligand.

Schematic representations of the functionalized gold nanoparticles are given in FIGS. 1a, 1b and 1c.

Biodegradable polymer means, in the sense of the invention, a polymer that will degrade or be absorbed naturally in a subject's body. The biodegradable polymer may also be called a bioabsorbable polymer. The biodegradable or bioabsorbable polymer particle may be designated hereinafter as polymer particle a/.

The polycation having a positive charge over a wide range of pH as defined above may be designated hereinafter as polycation c/. Said polycation c/ will always be located near the gold nanoparticles b/ since it is in electrostatic interaction with the latter. Thus, the polycation c/ may be encapsulated in the polymer particle a/ and/or adsorbed on the surface of the polymer particle a/.

The particulate structure of the invention is further characterized in that it comprises a surfactant adsorbed on the surface of the polymer particle a/. Said surfactant, when present, is therefore always present on the surface of the polymer particle a/ and is never encapsulated within the latter.

The presence of the surfactant is a function of the nature of the biodegradable polymer of the nanoparticle.
The surfactant of the invention is in particular polyvinyl alcohol (PVA) and/or a poloxamer, and is preferably PVA.
As examples of poloxamer, we may mention those marketed under the name Pluronic F-127 (Poloxamer 407), P85, L64.

Schematic representations of particulate structures of the invention with the surfactant are given in FIGS. 2a, 2b and 2c.

According to another aspect of the invention, the particulate structure further comprises at least one active principle encapsulated in the polymer particle a/, said active principle preferably being a chemotherapeutic agent and/or a fluorophor.

As an example of chemotherapeutic agent, we may mention temozolomide, paclitaxel, docetaxel and etoposide.

As an example of fluorophor we may mention indocyanine green (which is used in clinical practice for imaging) or other fluorophors such as cyanine 5, cyanine 7 or DiI (IUPAC name: “(2Z)-2-[(E)-3-(3,3-dimethyl-1-octadecylindol-1-ium-2-yl)prop-2-enylidene]-3,3-dimethyl-1-octadecylindole; perchlorate”).

Thus, according to the invention, the polymer particle a/ advantageously allows co-encapsulation of functional gold nanoparticles b/ and at least one active principle.

Schematic representations of particulate structures of the invention with the active principle are given in FIGS. 3a, 3b and 3c.

According to the invention, the macrocyclic chelating agents as mentioned above, which coat the gold nanoparticles, each comprise:
an anchoring function that comprises at least one sulfur atom for attaching the macrocyclic chelating agent to the gold nanoparticle, and which preferably comprises two sulfur atoms forming an endocyclic disulfide bond,
at least one complexation site of ions of interest and/or of radionuclides for medical imaging, said complexation site comprising at least one carboxylic acid function and/or an amine function,
a spacer arm located between the anchoring function and the complexation site or sites,
optionally a functionalization site allowing grafting of the chelating agent to an agent for targeting cancer cells.

Attachment between at least one sulfur atom of the anchoring function and the gold nanoparticle denotes more particularly an ionocovalent bond, which is a bond intermediate between a covalent bond and an ionic bond.

The macrocyclic chelating agent covering the gold particles is more particularly characterized in that:
the anchoring function is a radical selected from the group comprising:

[symbol]

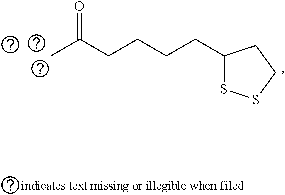

*—N—(CH₂—CH₂—SH)₂, *—C(═O)—(CH₂)ₙ—SH with n being an integer from 2 to 5 and mixtures thereof;
the spacer arm is a radical selected from the group comprising:
*—(CH₂)₂—CO—NH—(CH₂)₂—NH—*, *—NH—(CH₂—CH₂—O)ₘ—CH₂—CH₂—NH—* with m an integer equal to 0, 4 or 11, and mixtures thereof
the functionalization site, if present, is a radical, derived from an amino acid, selected from the group comprising:
*—NH—CH((CH₂)₄—NH₂)—CO—*, *—NH—CH(CH₂—OH)—CO—*,
*—NH—CH(CH—OH—CH₃)—CO—*, *—NH—CH(CH₂—C₆H₄—OH)—CO—*,
*—NH—CH((CH₂)ₙ—NH—*)—CO—* with n from 2 to 5, and mixtures thereof.
   As an example of amino acid from which the functionalization site is derived, we may mention lysine, serine, threonine, tyrosine.

According to one embodiment of the invention, the macrocyclic chelating agent is selected from the group comprising:

TADOTAGA, TANODAGA, TADFO, TA[DOTAGA-lys-NH₂], TA[NODAGA-lys-NH₂], TA[DOTAGA-lys-NODAGA] and mixtures thereof.
The meanings of these abbreviations are given hereunder.
DOTAGA: “1,4,7,10-tetraazacyclododecane-1-glutaric acid-4,7,10-triacetic acid”.
NODAGA: “1,4,7-triazacyclononane-1-glutaric acid-4,7-diacetic acid”.

### DFO: “Deferoxamine”.

TADOTAGA denotes the derivative of DOTAGA with the addition of a thioctic acid (TA) function.
TANODAGA denotes the derivative of NODAGA with the addition of a thioctic acid (TA) function.
TADFO denotes the derivative of DFO with the addition of a thioctic acid (TA) function.
TA[DOTAGA-lys-NH₂] denotes the derivative of TADOTAGA with the addition of an amine function via lysine.
TA[NODAGA-lys-NH₂] denotes the derivative of TANODAGA with the addition of an amine function via lysine.
TA[DOTAGA-lys-NODAGA] denotes a compound comprising a DOTAGA unit and a NODAGA unit joined together by lysine with addition of the thioctic acid (TA) function.

The organic layer surrounding the gold core, consisting of macrocyclic chelating agents, may be a “mixed” layer, which signifies that it consists of a mixture of macrocyclic chelating agents.

As examples of mixture we may mention [(TADOTAGA) (TANODAGA)], which denotes a mixture of TADOTAGA and TANODAGA, [(TADOTAGA) (TADFO)], which denotes a mixture of TADOTAGA and TADFO.

According to another embodiment of the invention:
the ion of interest for medical imaging, and more particularly for magnetic resonance imaging (MRI), is selected from the group comprising Gd3+, Ho3+, Dy3+ and mixtures thereof;
the radionuclide for medical imaging, and more particularly for nuclear imaging (SPET or PET), is selected from the group comprising ⁶⁴Cu, ⁸⁹Zr, ⁸⁸Ga, ¹¹¹In and mixtures thereof.
   Magnetic resonance imaging (MRI) is an imaging technique that allows three-dimensional visualization of biological tissues on the basis of the principle of nuclear magnetic resonance (NMR). MRI exploits the magnetic properties of the protons of water (major constituent of biological tissues, about 80%), which depend on the environment and therefore on the tissue.
   The nuclear imaging techniques require injection of radionuclides for carrying out functional imaging of the body. Two techniques may be distinguished: single-photon emission tomography—(SPET), which uses emitters of γ photons, and positron emission tomography (PET), which is based on the use of emitters of β⁺ positrons.

SPET and PET offer the advantage of having a very high sensitivity and of being able to perform functional imaging.

The functional gold nanoparticles b/, represented by Au@L(M), may therefore be followed by MRI (when M is an ion of interest), SPET or PET (when M is a radionuclide) and by X-ray imaging (owing to the gold).

The symbol @ denotes attachment or else ionocovalent bonding between the anchoring function of the macrocyclic chelating agent L and the gold nanoparticle.

The particulate structure of the invention is further characterized in that the polycation is selected from the group comprising polyethyleneimine (PEI), polylysine, polyarginine, polyamidoamine (PANAM), a poly(β-amino ester), chitosan and mixtures thereof, and is preferably polyethyleneimine. As a more particular example, we may mention branched (as opposed to linear) polyethyleneimine.

The term polycation is used because each of the compounds described above comprises amine groups, which may or may not be charged by protonation, depending on the pH. As already stated, the polycation used in the context of the invention has a positive charge over a wide range of pH, namely a pH range from 5 to 11.

According to another aspect, the biodegradable polymer of the particle is selected from the group comprising poly(lactic-co-glycolic) acid (PLGA), poly(lactic) acid (PLA), poly(glycolic) acid (PGA), polycaprolactone (PCL), a polyanhydride, the copolymers of each of said polymers with polyethylene glycol (PEG) and mixtures thereof, and is preferably PLGA or (PLGA-PEG) copolymer.

PLGA is a heterocopolymer of lactic acid and glycolic acid obtained by a copolymerization reaction. The monomers are joined together by ester bonds, giving a linear aliphatic polyester comprising x lactic acid units and y glycolic acid units. Thus, PLGA 75/25 identifies a copolymer whose composition is 75% lactic acid and 25% glycolic acid with a molecular weight between 7000 and 17000 g/mol. PLGA 50/50 is more particularly preferred.
PLGA is used in drug release on account of its excellent biocompatibility and biodegradability in lactic acid and glycolic acid, which are two monomers produced naturally in metabolic pathways.
As a guide, when the biodegradable polymer is the (PLGA-PEG) copolymer, the particulate structure of the invention does not comprise surfactant.

As a guide, the structural formulas of the macrocyclic chelating agents, polycations and biodegradable polymers are given in FIG. 4.

According to yet another aspect of the particulate structure of the invention, the macrocyclic chelating agent present on the surface of the gold nanoparticles is bound to an active agent targeting the integrins α_{V}β_{III} overexpressed on the tumor neovasculature, said targeting agent preferably being the cyclic RGD peptide.

Addition of a targeting agent makes it possible to achieve active targeting, in addition to passive targeting. The affinity of the biomolecule for the receptors overexpressed at the level of the tumor or the tumor neovasculature (in the case of RGD) will thus allow longer retention of the gold nanoparticles in the targeted zone.

The particulate structure is further characterized in that:
the hydrodynamic diameter of the polymer particle a/ is from 50 to 200 nm, preferably from 70 to 160 nm;
the hydrodynamic diameter of the gold nanoparticles b/ is from 3 to 15 nm, preferably from 6 to 10 nm.

The hydrodynamic diameter of a particle takes into account the diameter of the particle and of its so-called “hydration” layer.

In the present case, the hydrodynamic diameter of the polymer particle a/ is the diameter of the polymer particle a/ on whose surface the gold nanoparticles b/ and/or the surfactant are adsorbed.

In other words, the diameter of the polymer particle a/ with its layer formed by the gold nanoparticles b/ and/or the surfactant constitutes the hydrodynamic diameter of the polymer particle a/.
The diameter of the particulate structure is therefore equal to the hydrodynamic diameter of the polymer particle a/.
The hydrodynamic diameter of the gold nanoparticles b/ denotes the diameter of the gold nanoparticles covered on their surface with the macrocyclic chelating agents complexing at least one ion of interest and/or a radionuclide.

According to one embodiment of the invention, the particulate structure is more particularly characterized in that the gold nanoparticles b/ and optionally the active principle are encapsulated in the polymer particle a/, and said gold nanoparticles b/ may moreover optionally be adsorbed on the surface of the polymer particle a/.

The invention also relates to a method for preparing a particulate structure as defined above (i.e. in which the gold nanoparticles b/ (and optionally the active principle) are encapsulated in the polymer particle a/ and optionally adsorbed on the surface of the polymer particle a/). This method may be carried out by one or other of the two methods described below, and may be called “encapsulation process”.

Method 1

According to one embodiment, the method of the invention is characterized in that it comprises the following steps:
contacting an aqueous suspension of gold nanoparticles b/ with an aqueous solution of polycation, in order to obtain an assembly of gold nanoparticles b/ and polycation;
contacting the assembly of gold nanoparticles b/ and polycation as defined in the preceding step with a mixture of biodegradable polymer and water-miscible organic solvent, said organic solvent optionally being mixed beforehand with at least one active principle, in order to obtain a mixture of gold nanoparticles b/, polycation, biodegradable polymer and optionally active principle,
contacting the mixture of gold nanoparticles b/, polycation, polymer and optionally active principle as defined in the preceding step with water, optionally with an added surfactant, in order to precipitate the polymer in the form of particles around the gold nanoparticles b/ and optionally the active principle,
   the encapsulation yield of the gold nanoparticles b/ and optionally of the active principle in the polymer particles a/ is at least 75%, preferably at least 90%, and even more preferably at least 95%.

Method 2

According to another embodiment, the method of the invention is characterized in that it comprises the following steps:
contacting an aqueous solution of polycation with a mixture of biodegradable polymer and water-miscible organic solvent, said organic solvent optionally being mixed beforehand with at least one active principle,
contacting the assembly of polycation with the mixture of biodegradable polymer and organic solvent as defined in the preceding step, with the aqueous suspension of gold nanoparticles b/ in order to obtain a mixture of gold nanoparticles b/, polycation, biodegradable polymer and optionally active principle,
contacting the mixture of gold nanoparticles b/, polycation, polymer and optionally active principle, as defined in the preceding step, with water, optionally with an added surfactant, in order to precipitate the biodegradable polymer in the form of particles around the gold nanoparticles b/ and optionally the active principle,
   the encapsulation yield of the gold nanoparticles b/ and optionally of the active principle in the polymer particles a/ is at least 75%, preferably at least 90%, and even more preferably at least 95%.

As already stated, the active principle may be a fluorophor and/or a chemotherapeutic agent.

The encapsulation yield of the gold nanoparticles denotes the final weight of gold (i.e. the weight of gold encapsulated and optionally the weight of gold adsorbed) relative to the weight of gold used. In fact, during the encapsulation process it is possible that a proportion of the gold nanoparticles will not end up in the biodegradable polymer particle but will be adsorbed on the surface of the biodegradable polymer particle. The final weight of gold is identical to the weight of gold used if the encapsulation yield is 100%. This may nevertheless signify that a proportion of the gold nanoparticles ends up on the surface of the biodegradable polymer particle.

The encapsulation yield of the active principle denotes the weight of active principle encapsulated relative to the weight of active principle used. The active principle always end up inside the biodegradable polymer particle and never on its surface.

Regarding the biodegradable polymer, the final weight of polymer is identical to the weight of polymer used if the manufacturing yield is 100%.

The encapsulation rate (also called loading rate) of the gold nanoparticles denotes the final weight of gold (i.e. the weight of gold encapsulated and optionally the weight of gold adsorbed) relative to the weight of biodegradable polymer particles formed.

The loading rate of the gold nanoparticles obtained by the encapsulation process denotes the final weight of gold (i.e. the weight of gold encapsulated and optionally the weight of gold adsorbed) relative to the weight of biodegradable polymer particles formed.

The encapsulation rate of the active principle denotes the final weight of active principle (i.e. the weight of active principle encapsulated) relative to the weight of biodegradable polymer particles formed.

These are actual weights that are measured after formulation.

As a guide, the encapsulation rate of the gold nanoparticles is from 1 to 4%, preferably from 1 to 3%, and even more preferably about 1.4%.

The encapsulation rate of the active principle is from 0.5 to 5%, preferably from 1 to 3%, and even more preferably about 2%.

The method of preparation according to the invention described above (encapsulation process) advantageously gives an encapsulation yield of more than 75%, preferably at least 90%, and even more preferably at least 95%, whereas without using the polycation, the encapsulation yield, and the encapsulation rate, are zero.

The use of the polycation as defined above in the method of the invention advantageously makes it possible to obtain high encapsulation yields, i.e. close to 100%, or even equal to 100%, which is in particular a considerable benefit in terms of cost and time.

According to another embodiment of the invention, the particulate structure is more particularly characterized in that the gold nanoparticles b/ are adsorbed on the surface of the polymer particle a/, and the active principle, if present, is encapsulated in the polymer particle.

In this instance, the polymer particle a/ is a “filled” polymer particle a/, in which there is also optionally an active principle.

The invention also relates to a method for preparing a particulate structure as defined above (namely in which the gold nanoparticles b/ are adsorbed on the surface of the polymer particle a/ and the active principle, if present, is encapsulated in the polymer particle a/). This method may also be called “adsorption process”, and is characterized in that it comprises the following steps:
contacting a mixture of biodegradable polymer and water-miscible organic solvent, said organic solvent optionally being mixed beforehand with at least one active principle, with water, optionally with an added surfactant, in order to precipitate the biodegradable polymer in the form of particles, on the surface of which the surfactant is adsorbed, if it is present,
contacting the polymer particles a/ as defined in the preceding step with an aqueous solution of a polycation, in order to obtain polymer particles a/, on the surface of which the polycation is adsorbed, said biodegradable polymer particles moreover encapsulating the active principle if it is present,
contacting the polymer particles a/, on the surface of which the polycation as defined in the preceding step is adsorbed with an aqueous suspension of gold nanoparticles b/, in order to lead to the adsorption of the gold nanoparticles b/ on the surface of the polymer particles a/,
   the adsorption yield of the gold nanoparticles b/ on the surface of the polymer particle a/ is from 30 to 70%, preferably from 40 to 60%.

The adsorption yield of the gold nanoparticles denotes the final weight of gold (i.e. the weight of gold adsorbed) relative to the weight of gold used.

The loading rate of the gold nanoparticles obtained by the adsorption process denotes the final weight of gold (i.e. the weight of gold adsorbed) relative to the weight of biodegradable polymer particles formed.

Without the use of polycation, the adsorption yield would be zero.

The method of the invention also relates to the encapsulation process according to one of the two methods described above or the adsorption process described above, the common and original feature of these methods being the use of a polycation.

The loading rate obtained by the encapsulation process (i.e. gold nanoparticles inside the polymer particle and optionally on the surface of the polymer particle) is compared with the loading rate obtained with the adsorption process (gold nanoparticles only on the surface of the polymer particle), to show that there is indeed encapsulation, since the loading rate is different.

Schematic representations of the methods of preparation according to the invention are given in FIG. 5a (encapsulation process) and FIG. 5b (adsorption process).

According to an advantageous embodiment of the method of preparation according to the invention:
the aqueous solution of gold nanoparticles b/ is at a concentration from 8 to 12 grams of gold nanoparticles per liter of water,
the aqueous solution of polycation is at a concentration from 30 to 70 grams of polycation per liter of water,
the mixture of biodegradable polymer with the water-miscible organic solvent is at a concentration from 10 to 20 grams of polymer per liter of solvent, said organic solvent is selected from the group comprising dimethylsulfoxide (DMSO), dimethylformamide (DMF) and N-methyl-pyrrolidone,
the amount of active principle, if present, in the organic solvent is at a concentration from 0.15 to 0.75 grams of active principle per liter of solvent,
the amount of surfactant, if present, in water is from 5 to 10 grams of surfactant per liter of water.

These various concentrations or quantities are valid both for the encapsulation process and the adsorption process.

As already stated, the presence or absence of the surfactant depends on the nature of the biodegradable polymer used. Thus, when the biodegradable polymer is PEG or a (PLGA-PEG) copolymer, it is not then necessary to use a surfactant. When the biodegradable polymer is PLGA, the presence of the surfactant is necessary. The surfactant will be for example polyvinyl alcohol (PVA).

According to another advantageous embodiment of the method of preparation according to the invention, the polycation/ gold ratio, i.e. the ratio “aqueous solution of polycation/aqueous suspension of gold nanoparticles b/” varies from 4 to 8, and is preferably 5.

According to yet another advantageous embodiment of the method of preparation according to the invention, and more particularly of the encapsulation process, the pH of the aqueous solution of polycation varies from 9 to 11, and is preferably 10.8.

The pH of the solution of polycation has an influence on the size of the polymer particle obtained. A pH of 10.8 makes it possible to obtain polymer particles a/ with a hydrodynamic diameter of about 150 nm.

The original method of preparation according to the invention, which consists of using a polycation, leads to the production of particulate structures that have a monodisperse size, which can be modulated depending on the polycation/gold ratio and depending on the pH of the aqueous solution of polycation.

As a guide, the polydispersity index of the particulate structures must be less than 0.25. The particulate structures of the invention have a polydispersity index of about 0.16.

The polydispersity index represents the size distribution of a population of particles. The lower the index, the more the sample is monodisperse (uniform size). The conventional methods, which do not use polycation, lead to the production of particles that are polydisperse and/or generally of large size (of the order of a micrometer) and which comprise a low encapsulation yield.

Besides the production of particles of the order of a nanometer and which have a uniform size, the method of preparation according to the invention is also advantageous in that it is highly reproducible, both for the loading rate (encapsulation rate) obtained, and the size of the particles obtained.
The method of the invention is also advantageous in that it makes it possible to encapsulate gold nanoparticles b/, i.e. gold nanoparticles that are already functionalized, which in particular possess the contrast agent properties required for MRI, and optionally an active principle, with an encapsulation yield close to 100%, or even of 100%.

The main field of application of the particulate structures of the invention is imaging coupled with treatment of tumors by radiotherapy.

The biodegradable polymer particles a/, for example such as particles of PLGA, will perform the role of transporter, and the encapsulated and/or adsorbed gold nanoparticles b/ will perform the role of contrast agent and radiosensitizing agent. The first advantage of encapsulation (or adsorption) of the gold nanoparticles b/ in the (on the surface of the) polymer particles a/ is to increase the plasma half-life of the gold nanoparticles b/ in order to improve their tumoral accumulation and better exploit their radiosensitizing potential. As a guide, the plasma half-life of PLGA particles is 15 days. The gold nanoparticles b/ thus encapsulated and/or adsorbed circulate in the blood for a longer time and have the possibility of accumulating in larger amounts in the tumor. The improved tumoral accumulation of the gold nanoparticles makes it possible to increase the synergistic effect with radiotherapy. Moreover, once the polymer particles have degraded, the functionalized gold nanoparticles b/ return to the blood stream and can be eliminated rapidly by the renal route.

Moreover, the role of the bioabsorbable polymer particles a/ is not limited to the transport of the functionalized radiosensitizing gold nanoparticles b/. In fact, the polymer particles a/ allow, besides encapsulation of the gold nanoparticles b/, the encapsulation of at least one active principle such as a chemotherapeutic agent and/or a fluorophor.

This co-encapsulation endows the particulate structures of the invention with extremely interesting therapeutic properties, since the particulate structures of the invention make it possible to combine image-guided radiotherapy and chemotherapy and thus improve tumor treatment.

The particulate structures of the invention advantageously allow radiotherapy to be carried out in order to improve the effect of the therapy while reducing the side effects, in particular in the case of tumor treatment.

The invention also relates to a pharmaceutical composition containing a therapeutically effective amount of at least one particulate structure as defined above.

The amount of particulate structures may vary depending on the applications envisaged, and the patient's age and weight.

The particulate structures or pharmaceutical composition of the invention may be in a suitable form for administration by the intravenous route. As examples we may mention injectable suspensions.

The present invention also relates to a particulate structure as defined above, for use in the treatment of cancerous solid tumors.

The invention also relates to a method for therapeutic treatment of cancerous solid tumors comprising the administration of a therapeutically effective amount of at least one particulate structure or of a composition as defined above to a subject.

The present invention also relates to a particulate structure for use as defined above, by radiotherapy or chemotherapy, and more particularly by image-guided radiotherapy.

The invention also relates to a method of therapeutic treatment by radiotherapy or chemotherapy, and more particularly by image-guided radiotherapy, comprising administration of a therapeutically effective amount of at least one particulate structure or of a composition as defined above to a subject.

### BRIEF DESCRIPTION OF THE DRAWINGS

Other features, details and advantages will become clearer on reading the following detailed description, and on examining the appended drawings.

FIG. 1a is a schematic representation of the functionalized gold nanoparticles b/ in which the macrocyclic chelating agents are complexed to ions of interest.

FIG. 1b is a schematic representation of the functionalized gold nanoparticles b/ in which the macrocyclic chelating agents are complexed to a radionuclide.

FIG. 1c is a schematic representation of the functionalized gold nanoparticles b/ in which the macrocyclic chelating agents are complexed to ions of interest and a radionuclide.

FIG. 2a is a schematic representation of a particulate structure of the invention comprising a biodegradable polymer particle a/ in which 100% of the gold nanoparticles b/ are encapsulated. The gold nanoparticles form electrostatic interactions with the polycation.

FIG. 2b is a schematic representation of a particulate structure of the invention comprising gold nanoparticles b/ encapsulated in the biodegradable polymer particle a/ and adsorbed on the surface of the polymer particle a/.

FIG. 2c is a schematic representation of a particulate structure of the invention in which 100% of the gold nanoparticles b/ are adsorbed on the surface of the polymer particle a/.

A surfactant, adsorbed on the surface of the polymer particle a/, is represented in each of FIGS. 2a, 2b and 2c. The presence of the latter is optional, however, and each of these figures could also be represented without the surfactant.
The polycation is not shown with its positive charge in each of the particulate structures of the invention, so as not to complicate each of FIGS. 2a, 2b and 2c.

FIG. 3a is a schematic representation of a particulate structure of the invention corresponding to that in FIG. 2a but which further comprises an active principle encapsulated in the polymer particle a/.

FIG. 3b is a schematic representation of a particulate structure of the invention corresponding to that in FIG. 2b but which further comprises an active principle encapsulated in the polymer particle a/.

FIG. 3c is a schematic representation of a particulate structure of the invention corresponding to that in FIG. 2c but which further comprises an active principle encapsulated in the polymer particle a/.

The active principle is represented by a star in each of FIGS. 3a, 3b and 3c.

FIG. 4a shows the structural formulas of the macrocyclic chelating agents (L).

FIG. 4b shows the structural formulas of the polycations.

FIG. 4c shows the structural formulas of the biodegradable polymers.

FIG. 5a shows the method for preparing the particulate structures of the invention in which the gold nanoparticles b/ are encapsulated in the polymer particle a/, some of the gold nanoparticles b/ also being adsorbed on the surface of the polymer particle a/ (encapsulation process according to method 2).

FIG. 5b shows the method for preparing the particulate structures of the invention in which the gold nanoparticles b/ are adsorbed on the surface of the polymer particle a/ (adsorption process).

In the case when an active principle is added, the latter is mixed with the organic solvent and the biodegradable polymer, whether in the encapsulation process or in the adsorption process.
In these figures “Gold Np” denotes the gold nanoparticles.

FIG. 6 shows a transmission electron micrograph of a particulate structure of the invention, in which we can see a polymer particle a/ comprising several encapsulated and/or adsorbed gold nanoparticles.

FIG. 7 shows a blood kinetics graph showing the variation of the injected gold dose (as a percentage) per gram of blood as a function of time, for the gold nanoparticles alone (denoted by “Gold Np”), the gold nanoparticles encapsulated in PLGA particles (denoted by “NP3”) or in PLGA-PEG particles (denoted by “NP3-PEG”).

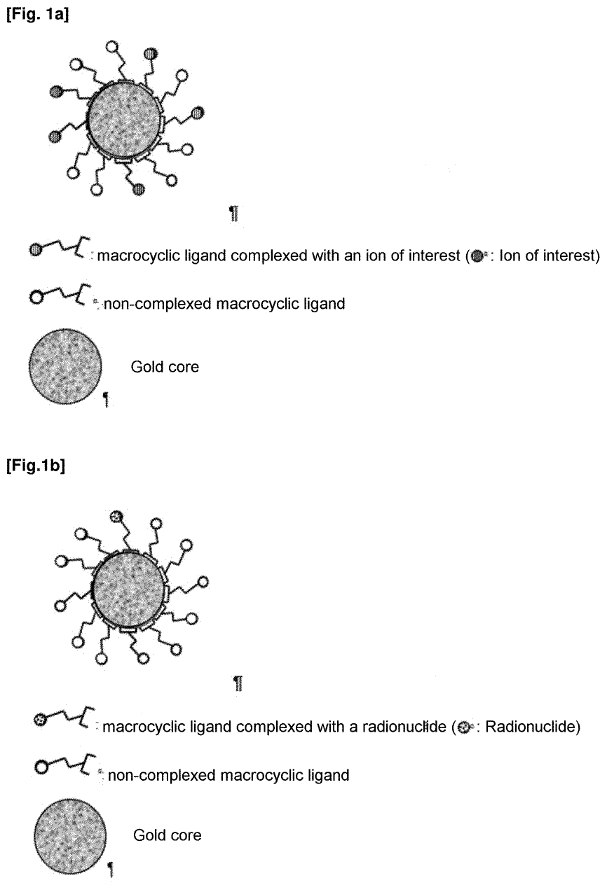

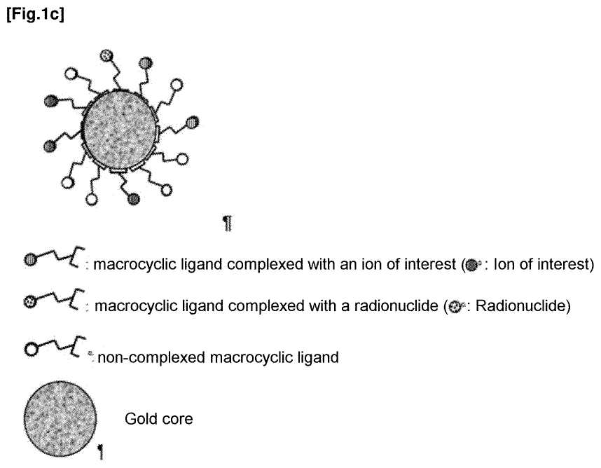

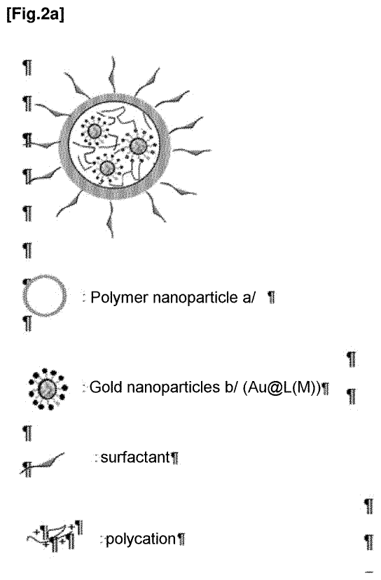

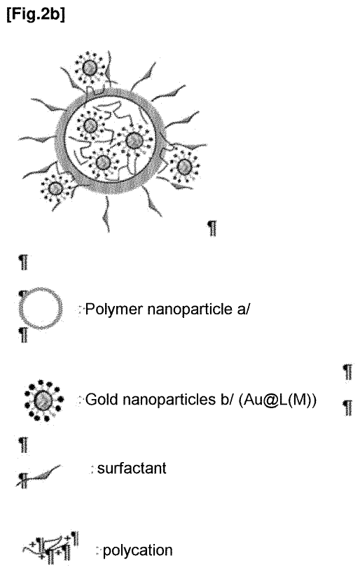

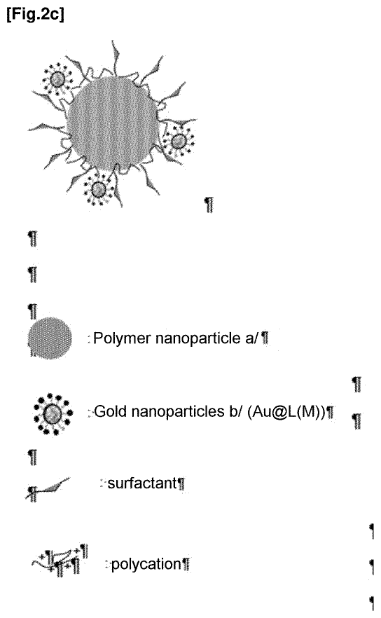

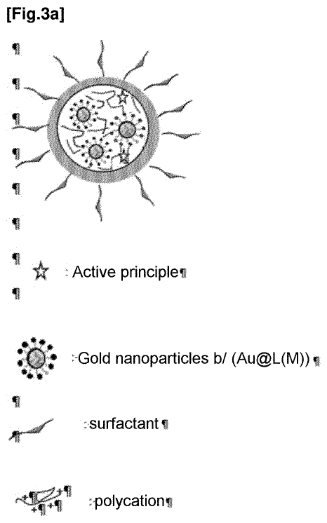

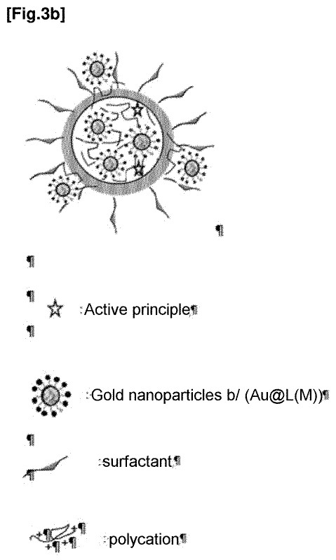

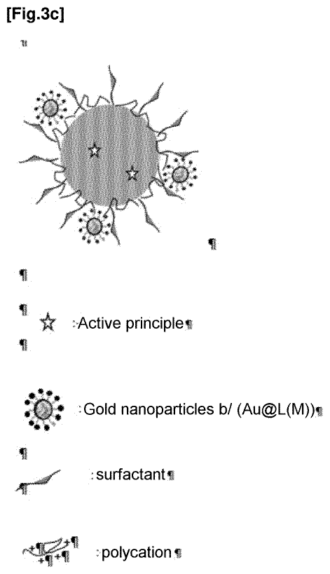

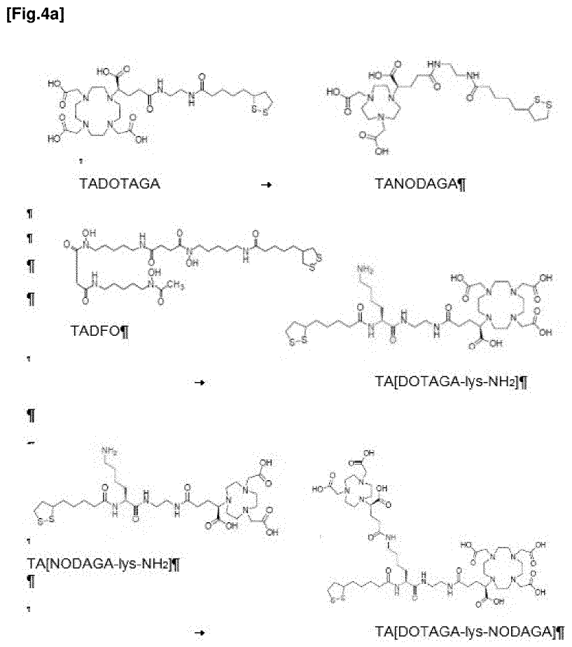

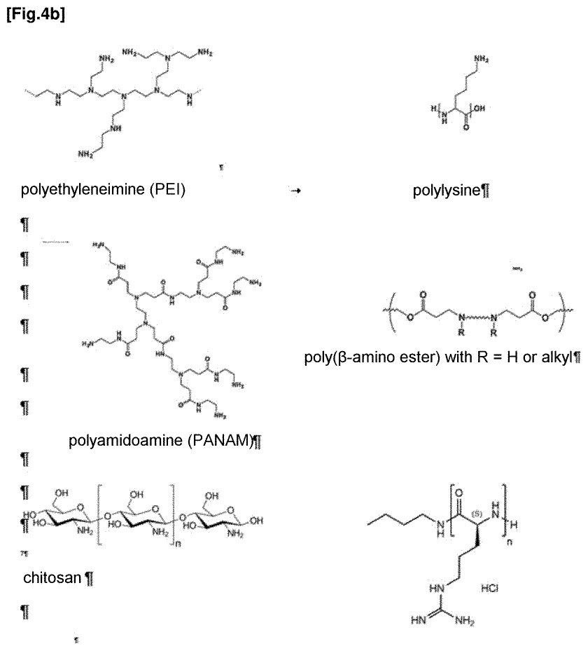

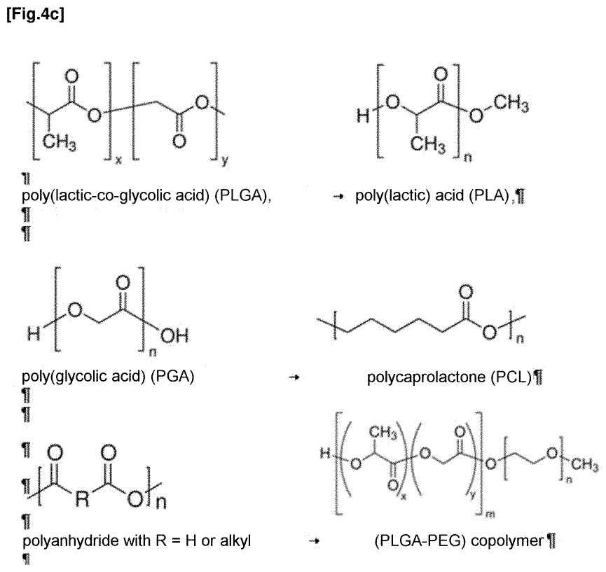

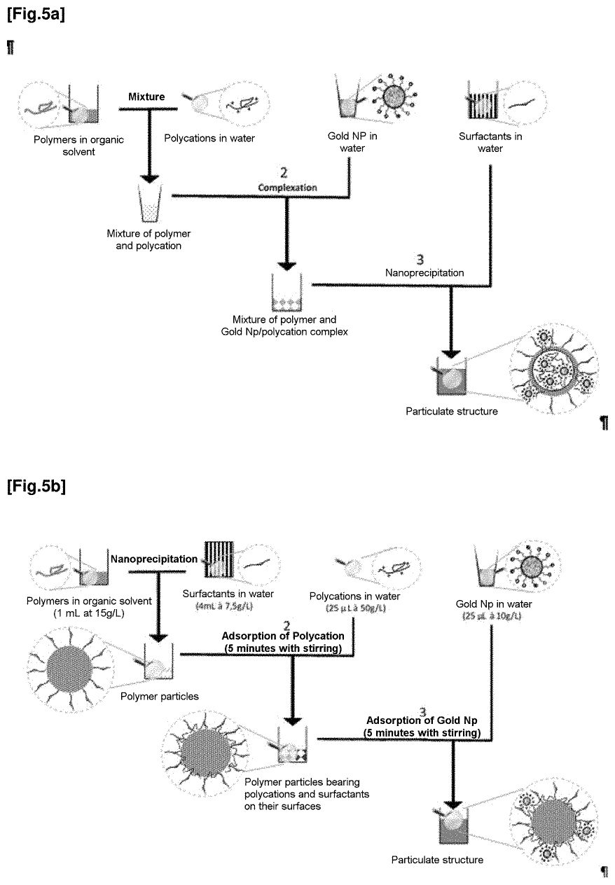

### DESCRIPTION OF THE EMBODIMENTS

### Examples

Preparation of particulate structures according to the invention in which:
the biodegradable polymer a/ is poly(lactic-co-glycolic) acid (PLGA) or a conjugate of poly(lactic-co-glycolic) acid and polyethylene glycol (PLGA-PEG),
the macrocyclic chelating agent is TADOTAGA and the ion of interest is gadolinium (Gd3+),
the polycation is polyethyleneimine (PEI).
   The surfactant is polyvinyl alcohol (PVA) and the water-miscible organic solvent is dimethylsulfoxide (DMSO).
   The gold nanoparticles b/, covered on their surface with the chelating agent TADOTAGA complexing the gadolinium ion, are represented hereinafter by:

### “Au@TADOTAGA(Gd)”.

Materials

More particularly, PLGA 50:50 (MW 7000-17000 Da) (marketed under the name Resomer® RG 502H) is obtained from Evonik Industries (Evonik Röhm GmbH) and PLGA-PEG 50:50 (PLGA: MW 25000 Da, PEG: MW 5000 Da) is obtained from Sigma Aldrich (St Louis, United States).

Chloroauric acid (HAuCl₄.3H₂O), sodium borohydride (NaBH₄), PVA (MW 30000-70000 Da), branched polyethyleneimine (PEI) (MW 25000 Da), gadolinium chloride (GdCl₃.6H₂O) and dimethylsulfoxide (DMSO) are obtained from Sigma Aldrich (Saint Louis, United States). The ligand TADOTAGA is obtained from Chematech (Dijon, France).

Synthesis of the Au@TADOTAGA(Gd) Nanoparticles

Synthesis of the gold nanoparticles is adapted from the single-phase protocol developed by Brust et al. (6). The gold nanoparticles are obtained by reduction of the gold salt (HAuCl₄.3H₂O) with NaBH₄ in the presence of the ligand TADOTAGA. Adsorption of TADOTAGA on the surface of the gold nanoparticles makes it possible to control the size and colloidal stability and allows immobilization of the gadolinium. More particularly, HAuCl₄.3H₂O (50 mg, 1.22×10−4 mol), dissolved in methanol (20 mL), is placed in a 250-mL round-bottomed flask. The ligand TADOTAGA (86 mg, 1.22×10⁻⁴ mol) in water (10 mL) is added to the solution of gold salt, with stirring. The mixture changes from yellow to orange. After some minutes, NaBH₄ (48 mg, 12.7×10−4 mol) dissolved in water (3 mL) is added to the mixture while stirring vigorously at room temperature. Stirring is maintained for 1 h. Then the mixture is dialyzed using a 6000-8000 kDa MWCO membrane.

To obtain the Au@TADOTAGA(Gd) final suspension ([Au]=51 mM, [Gd]=5 mM) before the process of encapsulation in the polymer particles, the gold suspension is concentrated and the gadolinium is trapped in the TADOTAGA chelating agent, stirring the suspension overnight with GdCl₃.6H₂O (370 μL at 135 mM for an Au@TADOTAGA(Gd) suspension at 10 mL). A gadolinium concentration of 5 mM guarantees stability of the suspension and an optimal MRI signal.

Synthesis of the PLGA or PLGA-PEG Polymer Particles Encapsulatinq Au@TADOTAGA(Gd)

The method for preparing the polymer particles encapsulating the gold nanoparticles b/ (Au@TADOTAGA(Gd)) is based on the method of nanoprecipitation by solvent displacement (13), but with the novel feature of using PEI. The inventors found that the size of the polymer particles can be modulated as a function of the PEI/ gold ratio and of the pH of the aqueous solution of PEI.

The inventors found in particular in the course of their research that a PEI/ gold ratio of 5 and a pH of about 10.8 were suitable for obtaining polymer particles having a hydrodynamic diameter of about 160 nm. In fact, a size of 160 nm±15 nm is advantageous in that it makes it possible to encapsulate a satisfactory amount of gold nanoparticles b/ while allowing a satisfactory manufacturing yield.

An aqueous solution of PEI (25 μL, 5% w/w) is mixed with 1 mL of solution of PLGA or of solution of PLGA-PEG in DMSO at 15 mg/mL and 18 mg/mL respectively.

1 N HCl is added to the aqueous solution of PEI beforehand in order to obtain a hydrodynamic diameter of the PLGA particles close to 160 nm±15 nm.

To modulate the PEI/ gold ratio for preparing different particles, only the concentration of the PEI is adjusted. The same volume of HCl is added to the solution as for preparation of the PLGA particles with a diameter of about 160 nm, independently of the concentration of PEI.

A suspension of Au@TADOTAGA(Gd) (25 μL, 10 mg/mL (i.e. 51 mM)) is added to the preceding solution comprising PEI and PLGA.

Then 4 mL of PVA dissolved in water at 0.75% is added gradually to the mixture, vortexed beforehand.

For preparing the PLGA particles by adsorption of the gold nanoparticles, the PLGA particles are formed beforehand according to the same protocol as the conventional PLGA particles.

Then a 5% solution of PEI (25 μL) is transferred to the suspension of PLGA particles with stirring. After incubation for 5 minutes, a suspension of Au@TADOTAGA(Gd) (25 μL, 10 mg/mL (i.e. 51 mM)) is finally added to the PLGA particles coated with PEI.

The various preparations are washed three times by ultracentrifugation at 30 000 g for 1 h, at 4° C. to remove the free gold nanoparticles. Finally the preparations are lyophilized using sucrose as cryoprotective, except in the batches used for determining the production yield, encapsulation yield and encapsulation rate.

These parameters are determined as follows:

$$Production yield ( \% ) = \frac{Quantity of PLGA particles formed}{Quantity of PLGA used} \times 100 \qquad ( 1 )$$

$$Encapsulation yield ( \% ) = \frac{Quantity of gold encapsulated and optionally adsorbed}{Quanitity of gold used} \times 100 \qquad ( 2 )$$

$$Encapsulation rate ( \% ) = \frac{Quantity of gold encapsulated and optionally adsorbed}{Quantity of PLGA particles formed} \times 100 \qquad ( 3 )$$

The various characteristics of the particles obtained in accordance with this protocol by varying the PEI/Gold ratio are described in Table 1 hereunder:

TABLE 1

| | | | | | NP3 | |
|---|---|---|---|---|---|---|
| Formulation | NP1 | NP2 | NP3 | NP4 | adsorbed | NP3-PEG |
| PEI/Gold | 0 | 6 | 5 | 4 | 5 | 5 |
| ratio | | | | | | |
| Hydrodynamic | 136 ± 4 | 135 ± 19 | 159 ± 14 | 196 ± 11 | 153 ± 3 | 198 ± 5 |
| diameter (nm) | | | | | | |
| Polydispersity | 0.05 ± 0.02 | 0.16 ± 0.03 | 0.16 ± 0.01 | 0.017 ± 0.03 | 0.007 ± 0.01 | 0.17 ± 0.03 |
| index | | | | | | |
| Production | 35 ± 5 | 54 ± 9 | 71 ± 7 | 82 ± 6 | 64 ± 3 | 54 ± 5 |
| yield | | | | | | |
| Encapsulation | 2 ± 2 | 102 ± 5 | 95 ± 8 | 88 ± 7 | 52 ± 7 | 86 ± 6 |
| yield | | | | | | |
| Encapsulation | 0.0 ± 0.0 | 1.5 ± 0.1 | 1.4 ± 0.2 | 1.3 ± 0.1 | 0.7 ± 0.1 | 1.1 ± 0.0 |
| rate | | | | | | |

We thus obtain particles having a hydrodynamic diameter from 130 nm to 200 nm (their size may be reduced further by adjusting the PEI/ gold ratio) with an encapsulation rate of about 1.4. The reduction in size leads inevitably to a reduction in production yield owing to the washing by centrifugation.

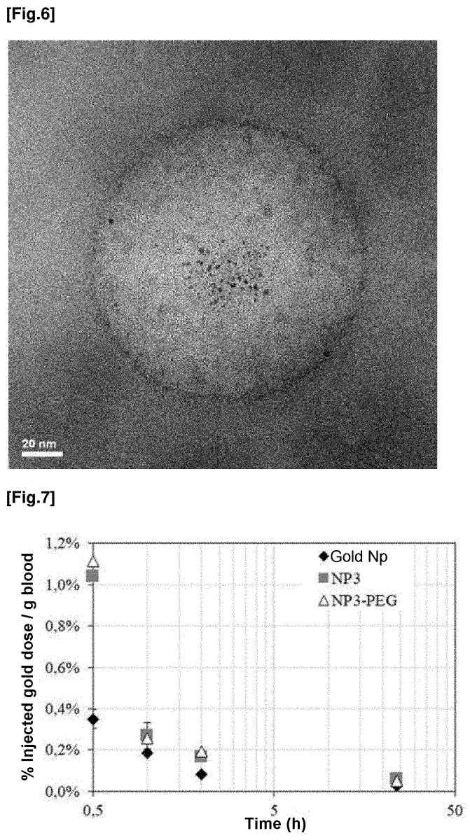

The NP3 particles (PEI/ gold ratio of 5) are selected for the tests in vivo. These particles represent a good compromise between size and production yield. The encapsulation rate is half as much in the case of the adsorption protocol (NP3 adsorbed) than the encapsulation protocol (NP3), which does indeed indicate encapsulation of the gold nanoparticles. The presence of gold is confirmed by imaging by transmission electron microscopy (see FIG. 6).

Image-Guided Therapy

The particulate structures of the invention are promising candidates for image-guided therapy if they display suitable behavior after intravenous injection: accumulation in the zone to be treated, absence of nanoparticles in the surrounding healthy tissues, preferential renal elimination (relative to the hepatobiliary route) and if the plasma half-life is increased relative to the gold nanoparticles.

Thus, a blood kinetic study was carried out on rats by injecting 500 μL of the NP3 suspension (or NP3-PEG) at 100 mg/mL in PLGA or an equivalent amount of gold of gold nanoparticles “alone” (Gold Np) by the intravenous route (penile vein) after isoflurane anesthesia. A blood sample was taken from the tail at different times and then the amount of gold present in the samples was measured by atomic absorption spectroscopy.

The results obtained are shown in FIG. 7.

### CONCLUSION

Encapsulation, whether carried out with PLGA or PLGA-PEG, increases the plasma half-life of the gold nanoparticles.

The encapsulation process of the invention advantageously allows encapsulation of gold nanoparticles within particles of reduced size (between 100 and 200 nm) with a yield close to 100% while maintaining a low polydispersity index. The particulate structure thus obtained makes it possible to increase the plasma half-life of the gold nanoparticles, and therefore has considerable, promising potential for improving the therapeutic effect of said gold nanoparticles.

The present invention is not limited to the examples described in the foregoing, only as examples, but includes all the variants that a person skilled in the art might envisage within the scope of protection sought.

### LIST OF DOCUMENTS CITED

### Nonpatent Literature

To all intents and purposes, the following nonpatent elements are cited:
(1) J. F. Hainfeld et al., Phys. Med. Biol., 49 (2004) N309-315;
(2) Gautier Laurent et al, Nanoscale, 8(2016) 12054-65;
(3) A. M. Gobin et al, Nano Lett., 7 (2007) 1929-1934;
(4) J. F. Hainfeld et al, Br. J. Radiol., 79 (2006) 248-253;
(5) K. T. Butterworth et al, Nanoscale, 4 (2012) 4830-4838;
(6) M. Brust et al, J. Chem. Soc. Chem, Commun., (1995) 0, 1655-1656;
(7) P. C. S. John Turkevich, Discuss Faraday Soc, 11 (n.d.) 55-75;
(8) Thesis of G. Laurent, Synthesis of multifunctional nanoparticles for image-guided radiotherapy. Organic chemistry. Franche-Comté University, 2014;
(9) T. Butterworth et al., Nanoscale, 2012; 4, 4830-4838;
(10) M. Yu et al., ACS nano, 2015, 9, 6655-6674;
(11) Wang Y et al., Biomed Opt Express, 2016, 7, 4125-4138;
(12) Luque-Michel et al., Nanoscale, 2016, 8, 6495-6506;
(13) H. Fessi et al., International Journal of Pharmaceutics, 1989, 55, R1-R4.

1.-18. (canceled)

19. A particulate structure, comprising:
a/ a biodegradable polymer particle,
b/ gold nanoparticles covered on their surface with macrocyclic chelating agents complexing at least one ion of interest and/or a radionuclide for medical imaging,
c/ a polycation having a positive charge over a pH range from 5 to 11,
the gold nanoparticles b/ being encapsulated in the polymer particle a/ and/or adsorbed on the surface of the polymer particle a/.

20. The particulate structure as claimed in claim 19, further comprising a surfactant adsorbed on the surface of the polymer particle a/, said surfactant preferably being polyvinyl alcohol (PVA) and/or a poloxamer.

21. The particulate structure as claimed in claim 19, further comprising at least one active principle encapsulated in the polymer particle a/, said active principle preferably being a chemotherapeutic agent and/or a fluorophor.

22. The particulate structure as claimed in claim 19, wherein the macrocyclic chelating agents that cover the gold nanoparticles each comprise:
an anchoring function that comprises at least one sulfur atom for attaching the macrocyclic chelating agent to the gold nanoparticle, and which preferably comprises two sulfur atoms forming an endocyclic disulfide bond,
at least one complexation site of ions of interest and/or of radionuclides for medical imaging, said complexation site comprising at least one carboxylic acid function and/or an amine function,
a spacer arm located between the anchoring function and the complexation site or sites,
optionally a functionalization site allowing grafting of the chelating agent with an agent for targeting cancer cells.

23. The particulate structure as claimed in claim 22, wherein:
the anchoring function of the macrocyclic chelating agent is a radical selected from the group comprising: [symbol] 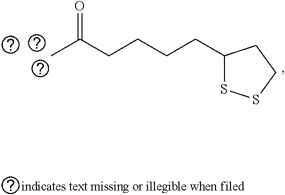
*—N—(CH₂—CH₂—SH)2, *—C(═O)—(CH₂)n-SH with n being an integer from 2 to 5 and mixtures thereof;
the spacer arm of the macrocyclic chelating agent is a radical selected from the group comprising:
*—(CH₂)2-CO—NH—(CH₂)2-NH—*, *—NH—(CH₂—CH₂—O)m-CH₂—CH₂—NH—* with m an integer equal to 0, 4 or 11, and mixtures thereof;
the functionalization site of the macrocyclic chelating agent, if present, is a radical derived from an amino acid, selected from the group comprising:
*—NH—CH((CH₂)₄—NH₂)—CO—*, *—NH—CH(CH₂—OH)—CO—*,
*—NH—CH(CH—OH—CH₃)—CO—*, *—NH—CH(CH₂—C₆H₄—OH)—CO—*,
*—NH—CH((CH₂)ₙ—NH—*)—CO—* with n from 2 to 5, and mixtures thereof.

24. The particulate structure as claimed in claim 19, wherein the macrocyclic chelating agent is selected from the group comprising: TADOTAGA, TANODAGA, TADFO, TA[DOTAGA-lys-NH₂], TA[NODAGA-lys-NH₂], TA[DOTAGA-lys-NODAGA]and mixtures thereof.

25. The particulate structure as claimed in claim 19, wherein:
the ion of interest for medical imaging, and more particularly magnetic resonance imaging (MRI), is selected from the group comprising Gd3+, Ho3+, Dy3+ and mixtures thereof;
the radionuclide for medical imaging, and more particularly nuclear imaging (SPET or PET), is selected from the group comprising ⁶⁴Cu, ⁸⁹Zr, ⁸⁸Ga, ¹¹¹In and mixtures thereof.

26. The particulate structure as claimed in claim 19, wherein the polycation is selected from the group comprising polyethyleneimine (PEI), polylysine, polyarginine, polyamidoamine (PANAM), a poly(O-amino ester), chitosan and mixtures thereof, and is preferably polyethyleneimine.

27. The particulate structure as claimed in claim 19, wherein the biodegradable polymer is selected from the group comprising poly(lactic-co-glycolic) acid (PLGA), poly(lactic) acid (PLA), poly(glycolic) acid (PGA), polycaprolactone (PCL), a polyanhydride, the copolymers of each of said polymers with polyethylene glycol (PEG) and mixtures thereof, and is preferably poly(lactic-co-glycolic) acid or [poly(lactic-co-glycolic) acid-polyethylene glycol] copolymer.

28. The particulate structure as claimed in claim 19, wherein the gold nanoparticles b/ are covered on their surface with a macrocyclic chelating agent bound to an active agent targeting the integrins α_{V}β_{III} overexpressed on the tumor neovasculature, said targeting agent preferably being cyclic RGD peptide.

29. The particulate structure as claimed in claim 19, wherein:
the hydrodynamic diameter of the polymer particle a/ is from 50 to 200 nm, preferably from 70 to 160 nm,
the hydrodynamic diameter of the gold nanoparticles b/ is from 3 to 15 nm, preferably from 6 to 10 nm.

30. The particulate structure as claimed in claim 19, wherein the gold nanoparticles b/ and optionally the active principle are encapsulated in the polymer particle a/, and said gold nanoparticles b/ may moreover optionally be adsorbed on the surface of the polymer particle a/.

31. The particulate structure as claimed in claim 19, wherein the gold nanoparticles b/ are adsorbed on the surface of the polymer particle a/, and the active principle, if present, is encapsulated in the polymer particle a/.

32. A method for preparing a particulate structure as claimed in claim 19, comprising the following steps:
contacting an aqueous suspension of gold nanoparticles b/ with an aqueous solution of polycation, in order to obtain an assembly of gold nanoparticles b/ and polycation;
contacting the assembly of gold nanoparticles b/ and polycation as defined in the preceding step with a mixture of biodegradable polymer and water-miscible organic solvent, said organic solvent optionally being mixed beforehand with at least one active principle, in order to obtain a mixture of gold nanoparticles b/, polycation, biodegradable polymer and optionally active principle,
contacting the mixture of gold nanoparticles b/, polycation, polymer and optionally active principle as defined in the preceding step, with water, optionally with an added surfactant, in order to precipitate the polymer a/ in the form of particles around the gold nanoparticles b/ and optionally the active principle,
the encapsulation yield of the gold nanoparticles b/ and optionally of the active principle in the polymer particles a/ is at least 75%, preferably at least 90%, and even more preferably at least 95%.

33. A method for preparing a particulate structure as claimed in claim 19, comprising the following steps:
contacting an aqueous solution of polycation with a mixture of biodegradable polymer and water-miscible organic solvent, said organic solvent optionally being mixed beforehand with at least one active principle,
contacting the assembly of polycation with the mixture of biodegradable polymer and organic solvent as defined in the preceding step, with the aqueous suspension of gold nanoparticles b/ in order to obtain a mixture of gold nanoparticles b/, polycation, biodegradable polymer and optionally active principle,
contacting the mixture of gold nanoparticles b/, polycation, polymer and optionally active principle as defined in the preceding step, with water, optionally with an added surfactant, in order to precipitate the biodegradable polymer in the form of particles around the gold nanoparticles b/ and optionally the active principle,
the encapsulation yield of the gold nanoparticles b/ and optionally of the active principle in the polymer particles a/ is at least 75%, preferably at least 90%, and even more preferably at least 95%.

34. A method for preparing a particulate structure as claimed in claim 19, comprising the following steps:
contacting a mixture of biodegradable polymer and water-miscible organic solvent, said organic solvent optionally being mixed beforehand with at least one active principle, with water, optionally with an added surfactant, in order to precipitate the biodegradable polymer in the form of particles, on the surface of which the surfactant is adsorbed if it is present,
contacting the polymer particles a/ as defined in the preceding step with an aqueous solution of a polycation, in order to obtain polymer particles a/, on the surface of which the polycation is adsorbed, said biodegradable polymer particles additionally encapsulating the active principle if it is present,
contacting the polymer particles a/, on the surface of which the polycation as defined in the preceding step is adsorbed, with an aqueous suspension of gold nanoparticles b/, in order to lead to adsorption of the gold nanoparticles b/ on the surface of the polymer particles a/,
the adsorption yield of the gold nanoparticles b/ on the surface of the polymer particle a/ is from 30 to 70%, preferably from 40 to 60%.

35. The method of preparation as claimed in claim 32, wherein:
the aqueous solution of gold nanoparticles b/ is at a concentration from 8 to 12 grams of gold nanoparticles per liter of water,
the aqueous solution of polycation is at a concentration from 30 to 70 grams of polycation per liter of water,
the mixture of biodegradable polymer with the water-miscible organic solvent is at a concentration from 10 to 20 grams of polymer per liter of solvent, said organic solvent is selected from the group comprising dimethylsulfoxide (DMSO), dimethylformamide (DMF) and N-methyl-pyrrolidone,
the amount of active principle, if present, in the organic solvent is at a concentration from 0.1 to 0.75 grams of active principle per liter of solvent,
the amount of surfactant, if present, in water is from 5 to 10 grams of surfactant per liter of water.

36. A method of treating cancerous solid tumors in a subject, comprising administering to a subject in need thereof a therapeutically effect amount of at least one particulate structure as claimed in claim 19.
